(19) United States
(12) Patent Application Publication
(10) Pub. No.: US 2024/0127580 A1
(43) Pub. Date: Apr. 18, 2024

(54) Optical Imaging System for Elliptical Polarization Discrimination Utilizing Multi-Spectral Pixelated Statistical Parametric Mapping

(71) Applicants: Ronald B. LaComb, West Greenwich, RI (US); Lanette Rachel Marie LaComb, West Greenwich, RI (US)

(72) Inventors: Ronald B. LaComb, West Greenwich, RI (US); Lanette Rachel Marie LaComb, West Greenwich, RI (US)

(21) Appl. No.: 18/137,814

(22) Filed: Apr. 21, 2023

Related U.S. Application Data
(60) Provisional application No. 63/334,695, filed on Apr. 26, 2022.

(51) Int. Cl. G06V 10/764; G01N 21/47; G01N 33/483; G06T 7/00; G06T 11/00; G06V 10/143

(52) U.S. Cl. CPC G06V 10/764; G01N 21/47; G01N 33/4833; G06T 7/0012; G06T 11/00; G06V 10/143; G01N 2021/4709; G06T 2207/10024; G06T 2207/30024; G06V 2201/03

(57) ABSTRACT

The present disclosure relates to a methodology and apparatus to measure the Stokes parameters pertaining to back scattered light resulting from an array of incident light beams mapping out the possible polarization states represented by the Point Care' sphere creating a multi-dimensional pixelated grayscale parametric data set which is used for algorithm development to classify or characterize substrates for structural signatures expressible by multi-wavelength back-scattered polarized light, caused by changes to tissue or material morphology, structural anomalies, material grains, disease, stress, pressure or temperature gradients, or other phenomena affecting signatures of back-scattered polarized light which may be regionally or locationally dependent. The optical polarization imaging apparatus features spinning optical elements consisting of linear polarizers and optical retarders to sequentially produce an array of illumination polarization beams at various wavelengths which are directed onto a target, the back scattered light is filtered by an analyzing optical circuit, containing spinning and stationary polarizers and retarders, a digital camera captures a series of filtered images which can be used to calculate the four Stokes parameters on a pixel by pixel basis for each of the incident polarizations mapping out the Point Care' Sphere, forming a data set consisting of normalized gray scale images pertaining to the four Stokes Vectors for each incident polarization, with one complete data set per input wavelength. The data set can be used to express depth and regionally dependent polarization descriptors (degree of circular polarization, degree of linear polarization, degree of polarization, polarization visibility) or used as an input to a machine learning based algorithm for classification on a pixel or pixel bin basis which can be used for cancer diagnostics, tumor demarcation or structural characterization of materials. The classified data can be overlayed with pictorial data creating a classification mask registered to physical coordinates of the target. Illumination of the target with an array of incident polarizations at various wavelengths optimizes regional structural alignment with one or more incident polarizations maximizing optical signatures for that region, analysis based upon the complete data set enables assemblies of regionally and polarization dependent description which can lead to more accurate regional and global classification of the target.

This application claims the benefit of U.S. Provisional Application No. 63/212,695. The entire disclosure of the above application is incorporated herein by reference.

### FIELD OF THE INVENTION

The present application relates to an optical imaging system and method for polarization discrimination of targets including biological tissues for cancer detection and demarcation and other materials for stress or other attribute. The system includes a multi-spectral light source, optical polarization state generation optics capable of producing sequentially a series of linear, circular and elliptical polarization states mapping out a subset of polarizations depicted by the Point Care' Sphere for illuminating a target, a camera capturing multiple images of the backscattered light off the substrate through a multi-state analysis optical circuit capable stepping through a series of optically filtered states to produce a set of gray-scale images enabling the calculation of the Stokes parameters for each pixel, and a computer for synchronization of data collection thereby producing a parametric data set consisting of normalized gray-scale images, one for each of the four Stokes parameters (S0,S1,S2,S3) for each illumination polarization state, one complete set per incident wavelength spectrum; an algorithm for data processing and statistical parametric mapping for establishing quantitative metrics. The classification resulting from quantitative analysis can be mapped to physical locations by overlaying the quantization values of each pixel with a digital picture of the target maintaining pixel registration. The present application relates to a means of forming a multi-spectral pixelated polarimetric parametric mapping of a target for quantitative characterization.

### BACKGROUND

This section provides background information related to the present disclosure. Conventional polarization based light imaging systems are based upon illuminating a substrate (tissue) with a light source of defined linear polarization state and analyzing the back scattered light through a cross-polarized filter producing pictures for qualitative analysis. In other configurations back scattered light is analyzed through Mueller matrices and or a subset of Stokes parameters, mostly characterizing the entire substrate by a single valued scalar classifier. Many approaches were developed for polarization discrimination techniques based upon these techniques varying light delivery and optical imaging techniques employing a plethora of microscopy techniques for improving image resolution, imaging filtering and staining/coloring techniques for qualitative analyze tissue/substrate characteristics and development of visual enhancement methods for cancerous tumor demarcation [U.S. Pat. No. 9,377,395 and US2015/0374276]. Previous optical polarimeter imaging approaches base analysis on a sub-set of polarization states illuminating a region of interest, and or collect a subset of polarization filtered data for various input wavelengths of illumination, to generate polarization filtered scattered picture data, and or treat the entire substrate as a single optical element described by a polarization metric with no regard to incident polarization alignment to structural organization of the target, and or are based upon qualitative visualization algorithms requiring color enhancement, statistical algorithms requiring large data sets to perform data characterization limiting the capability of polarization discrimination techniques.

### Objects and Advantage

Previous optical polarization discrimination techniques of targets rely upon harnessing wavelength based physiological discrimination and optical sectioning of a particular illumination polarization state and filtering the scattered light by an analysis filter cross-polarized to the incident polarization, forming data sets to be classified and analyzed qualitatively or by a single scalar classifier for the entire picture or target. Optical polarization discrimination of targets can be enhanced by classifying the target parametrically and by alignment of the illuminating polarization state with structural organizations of the target which may increase image contrast. Often targets possess many independent regions of structural organization, by varying the input polarization state to better align with regional structural features allows independent analysis of data images on a regional basis. Often how to align the input polarization with regional structural symmetries is unknown, this can be address by illuminating the target with a series of elliptical, linear and circular polarization states and filtering the captures images parametrically for regions of interest to assemble a more complete parametric mapping of target to form a reduced data set which includes regions of differing illumination polarization state. One example of structural organization of substrates is collagen formation in epithelial tissues in the presence of cancerous tumors. Healthy collagenous tissues are characterized by a random fibril organization intermixed with abundant nuclei. Epithelial cancers are known to cause collagen deformation, reducing the number of nuclei and reducing the overall randomness of fibral networks affecting the interaction of incident polarized light within the vicinity of cancerous tumors located in epithelial tissues. Current optical polarization imaging (OPI) techniques fail to take advantage of the localized organization of deformed collagen in the vicinity of an epithelial cancerous tumor supporting high contrast polarization signatures over limited regions of interest. Conventional Optical Polarization Discrimination techniques illuminate the entire substrate sequentially with one or more polarization states, analyzing scattered photons through cross polarizers without attempting to align input polarization illumination with structural organization of the tissue being analyzed, limiting classification to pictorial qualitative analysis dependent upon the pixel resolution of the imaging optics, or classification of the entire tissue by a single scalar value representing the degree of linear polarization or other parameter; Therefore, the illuminating polarization state may or may not be best aligned with localized collagen fibral networks, causing an averaging of the contrast ratio reducing the overall signal to noise level and effectiveness of the OP analysis motif.

The present disclosure provides a methodology and apparatus to utilize OPI as a quantitative parametric analysis motif on a pixel by pixel or pixel bin by pixel bin basis to take advantage of localized organization of structures influencing optical properties of scattered and attenuated light by better aligning the polarization of incident illumination with localized organizational structures of the probed target. In addition, the backscattered light is analyzed parametrically in lieu of pictorially to enable decoupling of the resolution of the data image upon which qualitative analysis is based, and photon—target interaction area enabling quantitative values to be assigned to pixels resulting from photon-target interactions taking place on a much smaller feature size comparted with the pixel resolution. Pixel registration is maintained with physical location of the target by registering pixels between parametric data files and corresponding digital pictorial images of the target enabling the quantitative mapping of the classified pixels to be overlayed with digital images linking quantitative descriptors to physical location of the target. Optical sectioning is accomplished by creating digital gray scale parametric data files at various wavelengths supporting a range of penetration depths into the target surface. The entire data file consists of a pixelated Stokes Vector (consisting of four pixelated normalized gray scale images S0ij,S1ij,S2ij,S3ij where i is the number of pixels rows and j is the number of pixel columns making up the gray scale image), for each illumination polarization state of a series of states mapping out the Point Care' Sphere pertaining to a wavelength spectral range of the source, additional data files are created from different source wavelengths. The complete data file set may contain several data sets each pertaining to unique source wavelengths. Numerical techniques are employed to develop classification techniques and algorithms for parametric data classification and description, algorithms may be physics related or developed by machine learning techniques or other technique proved to be effective by comparing predictions to Truth on a pixel or pixel bin basis.

The optical polarization discrimination technique employs a series of individual polarization states illuminating the sample, the polarization states form a discrete array mapping out the available linear, circular and elliptical polarization states as depicted by the Pointcare' sphere. A series of polarization based filtered images is taken of the backscattered light by a camera the combination of which is sufficient to calculate the Stokes parameters (S0,S1,S2,S3) on a pixel by pixel basis forming a Stokes parameter gray-scale representation of the back scattered light for each input illumination state. The Stokes parameter digital gray-scale images (S0ij,S1ij,S2ij,S3ij) are two dimensional data files (with i data rows and j data columns resulting in i*j or ij pixels) consisting of normalized scalar pixel values representing the individual Stokes parameter for each pixel for each incident polarization stated used to probe the surface. One way to generate polarization states mapping out the Point Care' Spere is by directing randomly polarized light through a combination of two rotating optical elements consisting of a linear polarizer LP and quarter wave plate QWP positioned in series; with each optical element independently stepping rotation through a series of discrete angle positions ranging from 0° to 180°. For example the LP is step from 0° to 180° sequentially by an integral number of in steps (N) of degrees, 18° (for N=10 steps) or 9° (for 20 steps), likewise while the QWP is stepped from 0° to 180° sequentially by an integral number of steps (K), by stepping the QWP through a series of steps varying from 0° to 180° each of angular extent 180/K degrees, the filtered light is polarized with N*K sequential polarization states mapping out the Point Care' Sphere. Spectral analysis involves repeating the data collection process at a number of illumination wavelengths λ1-λs (were s is the integral number of illumination wavelengths), which results in optical sectioning due to absorption and scattering mechanism associated with tissues, producing wavelength dependent penetration depths. The parametric data (including the Stokes gray-scale images and the data images making up the array of polarization filtered images used to calculate the Stokes data) can be used alone or in concert with pictorial data to create numerical algorithms based upon biophotonics, light tissue interactions, monte carlo ray analysis, polarization classifiers, degree of polarization, Stokes vectors, Mueller Matrices or other statistical analysis or combinations of techniques or developed through machine learning techniques.

Although the probing motif of this disclosure is general in principle, it has great potential toward optical discrimination of epithelial cancerous tissues. Collagenous networks in close proximity to cancer tumors express localized structural organization which can greatly affect scattering and attenuation of incident polarized light. By illuminating the target sample with an array of input polarization states at different input wavelengths allows localized regions of high contrast polarization signatures to be expressed within larger field of view parametric data files. Two dimensional Stokes based data files are generated for each input polarization are compared on a pixel by pixel or pixel bin by pixel bin for Stokes vector and input polarization state to develop classifier based algorithms by comparing parametric data to Truth images (allowing pixel registration with pixelated Truth digital images). Truth images can be formed on training sets by binary mapping of H & E stained slices of near by tissues, maintaining pixel registration with parametric data images. One technique to form a training set is to take a series of OPI images according the disclosed methodology, then Mohs surgery is performed and H & E stained tissues are used to produce a binary image representing health tissue as a logic “o” and cancerous tissue as a logic “1” on a pixel basis, enabling a pixel by pixel comparison of parametric data to Truth data enabling classification algorithms of parametric data to be developed. Utilizing of parametric algorithms can be used independently or in concert with other algorithms developed with alternative (pictorial or other) data sets to enhance the effectiveness of OPI discrimination of tissues.

### SUMMARY

This section provides a general summary of the disclosure, and is not a comprehensive disclosure of its full scope or all of its features.

The present disclosure relates to a method and apparatus to optically probe and classify the morphology of a target surface utilizing polarization discrimination. The method sequentially illuminates a surface with incident light of different wavelengths with an array of illumination polarization states mapping out a subset of the available polarization depicted by the Point Care's Sphere, the incident light back-scattered light emanating from the target is captured by a camera through a reconfigurable analysis optical circuit housing polarization optics capable of producing a series of filtered gray-scale images by a digital camera commensurate with the calculation of the individual Stokes parameters thereby forming a pixelated gray-scale parametric data set consisting of an array of two dimensional pixelated intensity images pertaining to each of the four Stokes parameters for each of the input polarization states and input wavelengths. Data analysis may include utilization of both pictorial and parametric pixelated data sets using the Stokes parameters or the data images used to make up the Stokes parameters with one or multiple classifiers, classifiers my include: statistical based classifiers, light transport based classifiers, machine learning based algorithms, parametric pixelated classifiers including degree of polarization, visibility of degree of polarization, absorption based classifiers, spectral classifiers, contrast based classifiers or other numerically derived classifier. Classification can be accomplished on a pixel by pixel, pixel bin by pixel bin or global basis. The advantage of utilizing a sequential array of illumination polarizations is due to alignment of polarized light with regionally organized structure producing high contrast localized optical signatures, assembly of high contrast regions from the multiple image data sets assembled establishes a more complete optical discrimination motif than that based upon illumination of a small number of incident global polarizations which may or may not be optimally aligned with structures producing high contrast signatures (effectively averaging out the contrast mechanism). Image collection commensurate with calculation of the Stokes parameters allows for quantitative analysis on a pixel by pixel, or pixel bin by pixel bin or global basis. One application is for epithelial cancer tumor detection (pertaining to skin cancers, ovarian cancer, colon cancer, throat cancer or other expressing tumors in epithelial tissues) and demarcation. Epithelial cancers are known to alter the assembly of collagen fibrils in the presence of tumors, the localized fibrillar assemblies interacts most strongly with polarized light aligned to the localized organization of the fibrillar assembly. Another application is for determination of polarization based electric dipole transitions associated with electron—atom interactions, which are know to be polarization dependent, this may be advantages for determination of protein structure analysis. Anther application for the determination of structural defects in objects, or pressure or temperature mapping of substrates.

One aspect the present disclosure relates to a method of polarization based discrimination of tissues forming a set of digital images utilized to calculate the four Stokes parameters forming a pixelated Stokes Vector parametric data set which is numerically processed through an algorithm to quantitatively discriminate between health and cancerous tissues. The apparatus consists of a light source capable of illumination at one or more wavelengths, the source light is filtered to illuminate the sample sequentially with an array of polarizations mapping out the Point Care' Sphere, the incident polarizations are created by passing the source light beam thru a spinning linear polarizer(Φ) and spinning retarder(ϕ), stepping each of the spinning optics through a series of incremental angles (180/N)° for Φ and (180/K)° for ϕ ranging from 0 deg to 180 deg thereby creating N*K unique polarization states ranging from linear to elliptical to circular polarization, discreetly mapping out the set of unique polarizations represented by the Point Care' Sphere. The optical elements can be rotated with respect to each other by housing them in rotational bearings connected to rotational motors incrementally stepping through a series of rotation angles varying between 0° and 180°. The incident light is scattered by the target tissue, the back scattered light is imaged by a digital camera through an analyzing optical circuit capable of producing a series of filtered images required to measure the four Stokes parameters S0,S1,S2,S3. The analyzing optical circuit consists of a stationary linear polarizer positioned in the horizontal configuration and a spinning retarder stepping a series of angles ranging from 0 to 180 degrees. The series of collected images are numerically processed to evaluate the individual Stokes parameters on a pixel or bin of pixel basis for each of the N input polarization. By stepping through a series of illumination wavelengths a multidimensional data set is produced. The data set is processed through an algorithm and numerically thresholded (setting all pixel intensity values to 1 which are above a scalar threshold set point value between 0 and 1, remainder of pixels are set to 0) to produce a final binary mask which can be overlayed with an optical image of the tissue to predict if and where cancerous tissue resides.

As one example of an algorithm the maximum back scattered intensity is found for each pixel or bin of pixels for the series of 4 Stokes parametric data sets forming one single two dimensional intensity based parametric picture for each of the 4 Stokes parameters, these data sets are used to calculate the degrees of polarization represented by the individual Stokes parameters S1,S2,S3 or the overall degree of polarization P=Sqrt(S1\hat{ }2+S2\hat{ }2+S3\hat{ }2)/S0 for each pixel. The final parametric pictures are classified to produce a binary mapping of a polarization state. In one example the data is used to form a gray scale image with the high intensity pixel represented by binary 0 intensity or “Black Pixel” while the low intensity pixels are represented by binary 1 or “White Pixel” forming a two dimensional mask which cab be overlayed with a registered pictures to map out which regions express high degrees of polarization which can be used to predict locations of collagen deformation. For evaluation, binary mappings can be compared to H & E stained samples of the tissue.

In another aspect of the present disclosure gray scale images are formed from an array of incident polarization states mapping out the Point Care' Sphere, with the back scattered light images through a series of analysis optics forming images which are commensurate with calculation of the Stokes parameters on a pixel basis, spectral analysis is used to generate Stokes images pertaining to each of the incident polarization states for an array of source wavelengths, enabling optical sectioning in addition to polarization discrimination of the target.

In another aspect of the present disclosure the optical circuit utilized to create a series of polarization states includes a light source lineally polarized to +45° which is filtered by two rotatable optical elements configured in series consisting of a waveplate (Φ) and rotator(ϕ) where ϕ, Φ represent the individual rotation angles which the elements are incrementally position varying form 0 to 180, where Φ is stepped by (180/N)° and is stepped by (180/K)° where both N and K are integers forming a total of N*K individual polarization states.

In another aspect of the present disclosure the optical circuit utilized to create a series of polarization states includes a polarized light source which light is filtered by a variable-phase wave plate, or Babinet-Soleil compensator, rotated between 0° to 180°, stepped by (180/N)° where N is an integers forming a total of N individual polarization states.

In another aspect the present disclosure the analyzing optical circuit consists of a rotating disc housing five aperture stations, the five apertures house an open aperture and four optical elements, a Horizontal polarizer, Vertical polarizer, a 45 degree polarizer, and a stacked element consisting of a quarter waveplate and 45 degree polarizer. The camera captures five images consisting of a control image and 4 filtered images, the four filtered images are used to calculate the four Stokes Parametric images, one set for each of the N input polarization states mapping out the Point Care' Sphere, and one complete N set per input wavelength.

In another aspect of the present disclosure the algorithm used to analyze the multi-dimensional data set is arrived at through machining learning techniques utilizing a training set of data including the individual polarization filtered pictures and parametric Stokes data compared with a Truth data set consisting of a binary mapping of a classification of the target maintaining pixel registration.

In another aspect of the present the data sets measured by the apparatus of this disclosure pertain to biological tissues for diagnosing cancerous tumors, the data set is used to train an algorithm by comparing to a Truth data set consisting of quantitative pixelated classification maps formed by thresholding H & E stained tissue pictures of nearby thinly sliced tissue (one tissue slice above or below), whereby evaluation is performed on a pixel by pixel or bin of pixel by bin of pixel basis, the resultant algorithm can be utilized independently or in conjunction with other algorithms developed using alternative data sets for tissue analysis including tumor detection and demarcation.

In another aspect of the present the data sets measured by the apparatus of this disclosure pertain to biological tissues analyzed for polarization and spectrally dependent energy absorption for determination of energy transitions and binding energies for classification of proteins and protein binding characterization associated with antibacterial activity, antimicrobial agents, and pharmacokinetics and pharmacodynamics of drugs.

In another aspect of the present disclosure wherein the target sample consists of materials other than biological tissues, including structural materials, plastics, glass, composite materials or other materials or objects which produces polarization dependent scattering signatures analyzed for stress or other physical or physiological property according to the apparatus and methodology of this disclosure. Polarization discrimination can consist of utilizing an array of illumination polarizations and wavelengths commensurate with target penetration which may not be visible.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure. The accompanying drawings, which are incorporated into and constitute a part of the specification, illustrate specific embodiments of the apparatus, systems, and methods and, together with the general description given above, and the detailed description of specific embodiments serve to explain the principles of the apparatus, systems, and methods.

In the Drawings:

FIG. 1A shows a system diagram for probing a target surface utilizing an array of source polarizations at different wavelengths and detection methodology measuring the back scattered Stokes parameters on a pixel by pixel basis.

FIG. 1B shows a system diagram of an implementation of system components in FIG. 1A with a rotating disc housing optics for image collection and measurement of pixelated Stokes parameters.

FIG. 1C shows an array of the input polarization states mapping out the Point Care' Sphere.

FIG. 1D shows an image of the collagen structure of ovarian tissue for early stage cancer, the fibrils are shown to form localized organizational assemblies best aligned to unique polarization states.

FIG. 2A shows a method of calculating the Stokes parameters from a series filtered digital images captured by the apparatus of FIG. 1A.

FIG. 2B shows a method of calculating the Stokes parameters from a series of filtered digital images captured by the apparatus of FIG. 1B.

FIG. 3 shows a method of classifying the data set by degree of polarization for multiple wavelengths.

FIG. 4 shows a tissue slice taken during Mohs surgery and H & E stained, and a binary mapping illustrating regions of cancerous tissues and healthy tissue, the image serves as Truth for algorithm development and data analysis.

FIG. 5 Shows a block diagram of a methodology to collect a complete pixelated data set utilizing an apparatus pertaining to this discloser and algorithm development employing machine learning techniques.

### DETAILED DESCRIPTION

Example embodiments will now be described more fully with reference to the accompanying drawings.

Referring to the drawings, to the following detailed description, and to incorporated materials, detailed information about the apparatus, systems, and methods is provided including the description of specific embodiments. The detailed description serves to explain the principles of the apparatus, systems, and methods described herein. The apparatus, systems, and methods described herein are susceptible to modifications and alternative forms. The application is not limited to the particular forms disclosed. The application covers all modifications, equivalents, and alternatives falling within the spirit and scope of the apparatus, systems, and methods as defined by the claims.

The present invention includes a randomly polarized optical source capable of producing one or more optical wavelengths, rotating polarization optics including linear polarizers and retarders to produce an array of polarizations illuminating a substrate, a cameral collecting images through rotating polarization optics to filter backscattered light to measure the Stokes parameters pertaining to each of the incident illumination polarization forming a array of pixelated parametric data for each incident wavelength, data is analyzed by a algorithm for a classifier of interest.

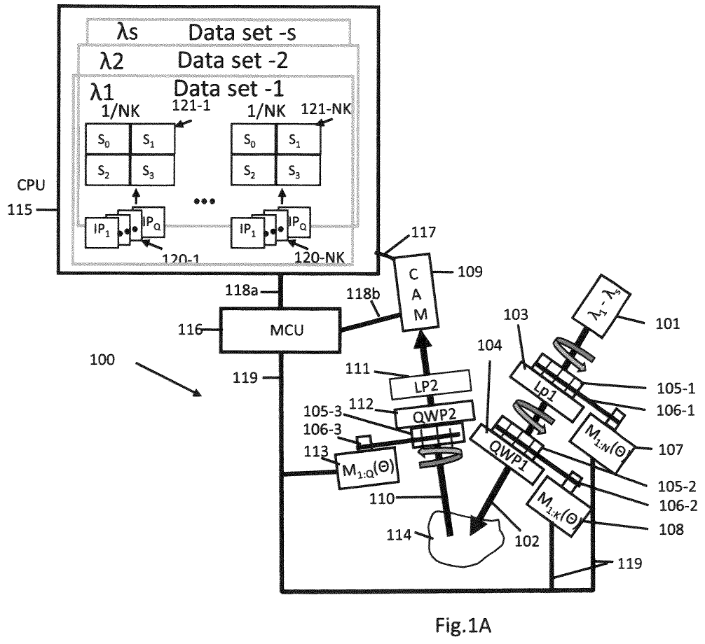

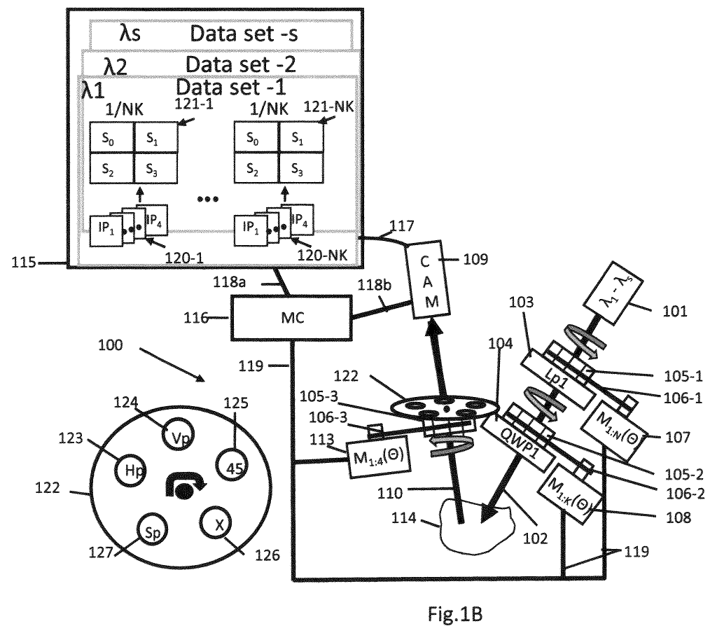

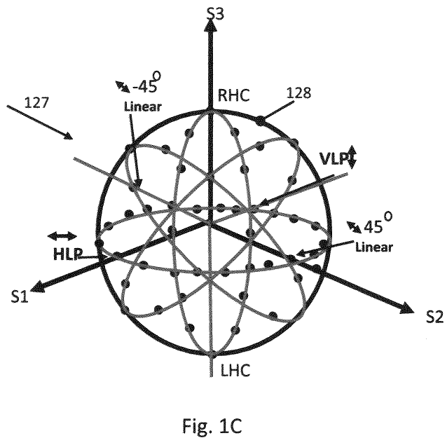

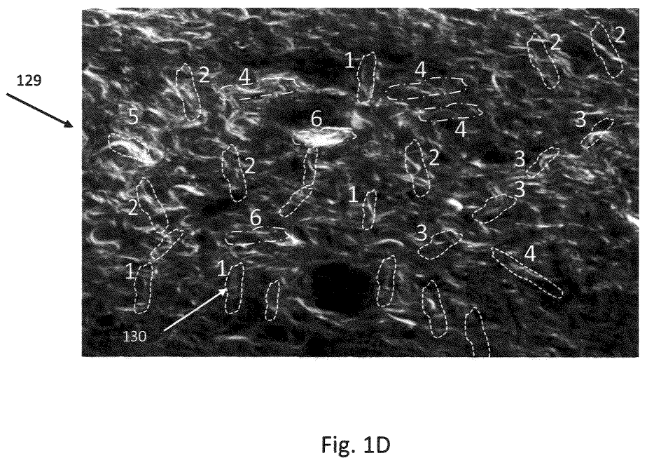

Referring to FIG. 1A an apparatus 100 for polarization discrimination of substrates is shown pertaining to this disclosure. The apparatus includes: an optical light source 101 capable of generating randomly polarized light at one or more wavelengths λ1-λs (integer s), a method for producing an array of incident polarizations 102 upon a target 114, the source light is sequentially propagated through a rotating linear polarizer 103 Lp1 housed by rotational bearing 105-1 turned by a motor 107 and transmission coupler 106-1 consisting of a belt or gear, followed by a rotating quarterwave plate QWP1 104 housed by a rotational bearing 105-2, rotated by motor 108 and transmission coupler 106-2, back scattered light 110 is filtered by a second rotating quarter waveplate QWP2 112 housed by a bearing 105-3, motor 113 and transmission coupler 106-3 followed by a stationary linear polarizer LP2 111, and imaged by a digital camera CAM 109. The motor 107 is stepped N (2 or more) steps varying polarization from horizontal to vertical, motor 108 is stepped K (2 or more) steps varying the QWP through a series of angles ranging from 0 to 180 degrees, QWP2 is rotated by Motor 113 a series of Q (8 or more) steps of angles ranging from 0 to 180 degrees, Motor controls are accomplished by a motor controller unit MCU 116 and computer CPU 115, an array of Q (IP₁-IP_{Q}) digital images 120-1 through 120-NK are taken for each of the N*K input polarizations for calculating the Stokes Parameters S0,S1,S2,S3 on a pixel basis with integral i rows and integral j columns forming gray scale images S0ij,S1ij,S2ij,S3ij consisting of a total of i*j pixels, thereby forming a data set consisting of N*K sets of Stokes Vectors Sv=(S0,S1,S2,S3) 121-1 through 121-NK, for each of the NK input polarizations, one complete Data set per source wavelength λ1-λs, data collection and motors are controlled by a computer CPU 115 and motion control unit MCU 116 connected by cables 117,118 and 119.

Referring to FIG. 1B a polarization discrimination apparatus according to this disclosure is shown 100 with a rotating disc optical analysis filter deck 122 containing five optical ports 123-127 which sequentially rotates bringing each portal in line with the camera's 109 field of view to filter backscattered illumination from the target 114 sequentially through an array of polarization optics, the disc is rotated to each of the ports by motor 113, with port 123 housing a linear polarization filter in the horizontal position (Hp), port 124 housing a vertical linear polarization filter (Vp) in the vertical position, port 125 housing a linear polarization filter in the 45 degree position (45), port 126 housing an empty aperture (x) for taking a calibration picture of the tissue 114 and port 127 housing a stacked polarization optic (Sp) containing a 45 degree linear polarizer and a quarter waveplate, the camera 109 captures an image of the target though each of the four disc portals housing polarization optics collecting a series of four images 120 (IP1-IP4) for each NK input polarizations defined by the rotation of Motors 107 (taking N steps) and 108 (taking K steps) controlled by the motion controller MC 116. The data set 121 (one per each source wavelength (λ1-λs)) is analyzed by the computer CPU 115 using algorithmic techniques to classify the substrate being analyzed on a pixel by pixel or pixel bin or global bases.

Referring to FIG. 1C show an illustration of the elliptical polarization states represented in the Point Care', sphere with the Stokes parameters S1,S2,S3 forming the axis, with HLP referring to horizontal linear polarization VLP referring to vertical linear polarization 45 degree referring to 45 degree linear polarization, RHC referring to right hand circular polarization, LHC referring to left hand circular polarization, the array of illumination polarizations mapped out by the rotating motors 107 and 108 are signified by the dots 128, where the total number of individual polarization states 128 total N*K.

Referring to FIG. 1D, a second harmonic generation image 129 of precancerous ovarian cancer, the images shows the formation of localized organizational fibrillar structures which are best aligned with different polarization states, 6 different polarization states consisting of rotated linear polarization are illustrated by dashed lines 130 (1-6) depicting regions of strong alignment pertaining to high contrast polarization based optical signatures, assembly of high contrast data pertaining to different target illumination polarizations is used for data set assembly and subsequent algorithm development for substrate classification.

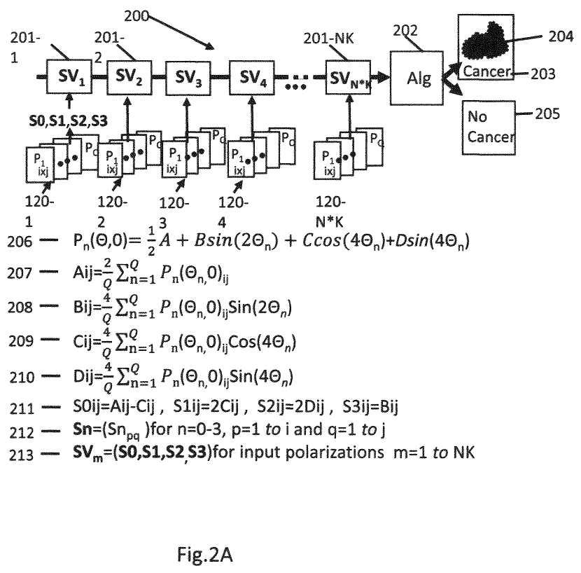

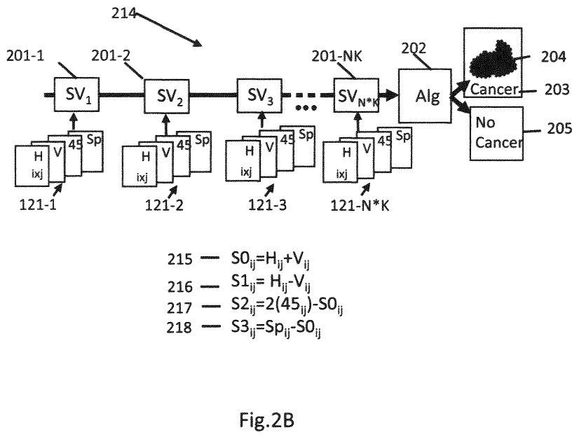

Referring to FIG. 2A shows a method 200 of calculating the Stokes parameters S0,S1,S2,S3 forming the Stokes Vectors SV=(S0,S1,S2,S3) for each of the N*K input illumination polarization forming a data set SV₁-SV_{NK} 201-1-201-NK which is processed by an algorithm 202 to classify a tissue as cancer 203 of healthy 205, and if cancerous a mapping of the location of cancerous tissues 204 from a series filtered digital gray scale images containing i rows and j columns of pixelated data captured by the apparatus of FIG. 1a, where the apparatus of FIG. 1a captures a series of Q pictures 120 (P₁-P_{Q}) for each of the N*K input polarizations illuminating the target 114, forming a set of gray scale picture arrays 120-1 through 120-NK each with Q pictures pertaining to the individual Q rotational angles of the QWP2 112 filtering the reflected light 110 sensed by the Camera 109, the Stokes parameters are calculated following the summation of each Q data array following equation for Pn 206, evaluating Aij 207, Bij 208, Cij 209 and Dij 210 for each of the i*j pixels, with Soij,S1ij,S2ij,S3ij evaluated pixel by pixel by equation 211, forming intensity images S0-S1,S2,S3 each containing ij pixels according to equation 212 forming vectors SVm=(So,S1,S2,S3) for each of the m input polarizations ranging m=1 to NK.

Referring to FIG. 2B shows a method 200 of calculating the Stokes vectors SV₁-SV_{NK} using the data set of digital gray scale images 121 taken by apparatus FIG. 1B for each of the NK input polarizations, The four digital pictures 121 taken for each input polarization refer to gray scale pictures of i*j number of pixels taken which are filtered sequentially by horizontal filter 123 forming picture H, linear vertical polarization filtered picture V taken via vertical linear polarization filter Vp 124, 45 degree filtered digital picture 45 taken via 45 degree linear polarization filter 125 and stacked digital picture Sp taken via stacked polarization optics 127, these digital pictures are used to calculate the pixelated Stokes parameters S0ij by equation 215, S1ij by equation 216, S2ij by equation 217 and S3ij by equation 218, the combination of these parameters forming Stokes vectors SVₘ for each of the m input polarization states ranging from 1 to NK.

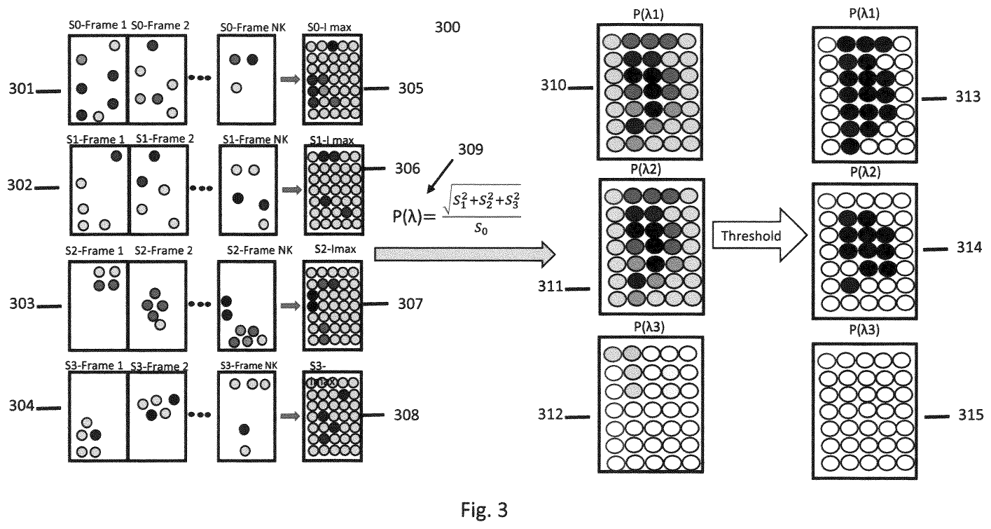

Referring to FIG. 3, a methodology 300 is shown to classify a data set generated by the apparatus of this disclosure pertaining to a input wavelength λ utilizing the degree of polarization P(λ) as the classifier, for each gray scale image Frame (from 1 to NK) representing a Stokes parameters S0—Frame #301,S1—Frame #302, S2—Frame #303, S3—Frame #304 the maximum intensity value is found for each pixel ij (or bin of pixels—illustrated as shaded dots) for all the NK Frames producing pixelated images S0—Imax 305, S1—Imax 306, S2—Imax 307, S3—Imax 308, where black dots represent high level of polarization, and white dots low level of polarization, for example S0-Imax contains the maximum gray scale intensity value for each pixel (or bin of pixels) among the NK Frames of Stokes parameter S0, utilizing the Imax data files for each of the Stokes parameters S0—Imax, S1—Imax, S2—Imax, S3 I-max the degree of polarization P(λ) is calculated using equation 309 forming a single data P(λ1) 310 for each input wavelength A, optical sectioning is accomplished by repeating the process at alternative wavelengths supporting different penetration depths into the target producing degree of polarization images P(λ2) 311 and P(λ3) (here 3 wavelengths were assumed but this number is arbitrary) intensity thresholding is used to convert the 8-12 bit intensity image to a binary image predicting where a cancer is present (black pixels) and where healthy tissue is present (white pixels) allowing for cancer screening and demarcation aiding in surgical removal of cancerous tissue.

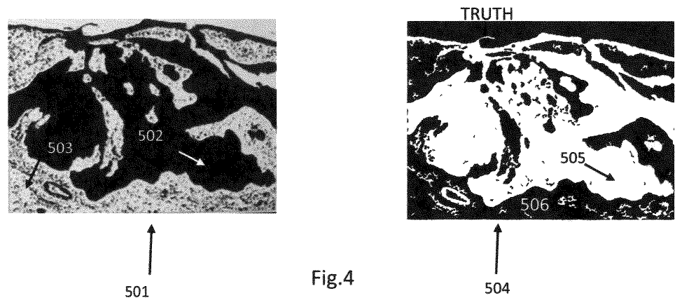

FIG. 4 shows a picture of a H & E stained cancerous tissue slice 501 taken during Mohs surgery illustrating cancerous regions as dark (purple) pigmentation 502 and healthy tissue as light (pink) pigmentation regions 502, a TRUTH quantitative binary image 504 is developed by intensity thresholding the H & E stained picture to represent cancerous tissue 505 as White (intensity=1) and healthy tissue as Black (intensity=) 506, the Truth image (pixelated quantitative digital image) is used to compare with pixelated classifiers derived from the collected data set for algorithm development and pixel classification.

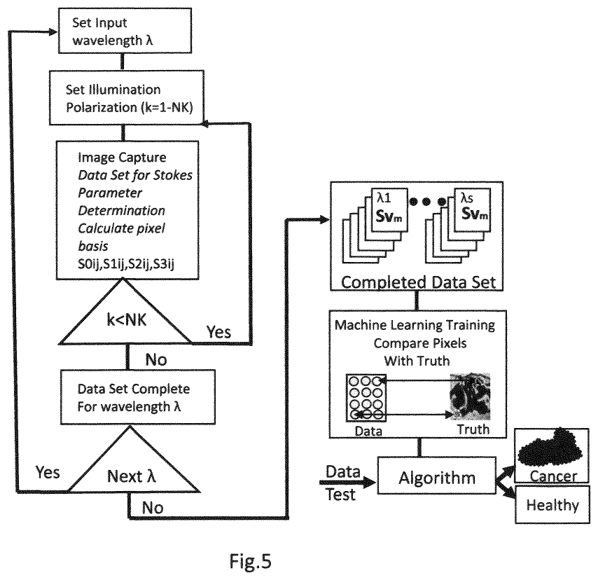

FIG. 5 shows a block diagram of a methodology to collect a complete data set consisting of NK sets of pixelated Stokes Vector parametric images S0ij,S1ij,S2ij,S3ij per input wavelength λ1-λs (were s is the number of individual wavelengths) where the completed data consisting of NK sets of the four parametric Stokes parameters forming the Stokes Vector Svₘ (m=1 to N*K) set is compared to a truth image pertaining to FIG. 4 to classify each pixel or pixel group as cancerous or healthy thereby training an algorithm by machine learning techniques, the resultant Algorithm can be used to test additional data sets pertaining to tissues being analyzed to predict if the sample contains cancerous tissues and if so where they reside on a pixel by pixel or pixel bin basis.

Although the description above contains many details and specifics, these should not be construed as limiting the scope of the application but as merely providing illustrations of some of the presently preferred embodiments of the apparatus, systems, and methods. Other implementations, enhancements and variations can be made based on what is described and illustrated in this patent document. The features of the embodiments described herein may be combined in all possible combinations of methods, apparatus, modules and systems. Certain features that are described in this patent document in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a subcombination or variation of a subcombination. Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described above should not be understood as requiring such separation in all embodiments.

Therefore, it will be appreciated that the scope of the present application fully encompasses other embodiments which may become obvious to those skilled in the art. In the claims, reference to an element in the singular is not intended to mean “one and only one” unless explicitly so stated, but rather “one or more.” All structural and functional equivalents to the elements of the above described embodiments that are known to those of ordinary skill in the art are expressly incorporated herein by reference and are intended to be encompassed by the present claims. Moreover, it is not necessary for a device to address each and every problem sought to be solved by the present apparatus, systems, and methods, for it to be encompassed by the present claims. Furthermore, no element or component in the present disclosure is intended to be dedicated to the public regardless of whether the element or component is explicitly recited in the claims. No claim element herein is to be construed under the provisions of 35 U.S.C. 112, sixth paragraph, unless the element is expressly recited using the phrase “means for.”

While the apparatus, systems, and methods may be susceptible to various modifications and alternative forms, specific embodiments have been shown by way of example in the drawings and have been described in detail herein. However, it should be understood that the application is not intended to be limited to the particular forms disclosed. Rather, the application is to cover all modifications, equivalents, and alternatives falling within the spirit and scope of the application as defined by the following appended claims.

What is claimed is:

1. A apparatus for optically probing substrates using a system containing a light source capable of producing randomly polarized light beam at various wavelengths, a multi-state polarizing optical circuit filtering said light beam producing a series of individual polarization states consisting of linear, circular and elliptical polarizations discreetly mapping out the Point Care' Sphere for illuminating a target, a multi-state analysis optical circuit capable of producing a discrete series of filtered images enabling the calculation of Stokes parameters on a pixel basis forming two dimensional gray scale images for each four Stokes parameters (S0ij,S1ij,S2ij,S3ij) for each said polarization state, a camera taking images of said illuminated target, a computer (CPU) and motor control unit (MCU) synchronizing data collection producing a plurality of digital gray scale parametric images forming a data set for each source wavelength. Data analysis is performed on the entire parametric data set through a numerical algorithm or numerical technique based upon biophotonic principles developed to optimize quantitative target classification on a pixel basis registered to physical location or data coordinates of the target.

2. The apparatus of claim 1 wherein said multi-state polarizing optical circuit filtering said light beam consisting of two rotatable optical elements positioned in series producing a resultant light beam possessing a series of independent polarization states pertaining to the individual incremental rotational angles of said optical elements;
whereby the first optical elements is a linear polarizer and second optical element is a quarter waveplate, where by both optical elements are positioned in rotational bearings capable of being individually rotated by independent motors to incremental rotation angles controlled by said computer and motor control unit, whereby a series of polarization states are produced by rotating the linear polarizer incrementally from 0° to 180° by an integral number of steps N, for each of the N incremental angular positions the quarter wave plate is incrementally rotated K steps ranging from 0° to 180° forming N*K individual multi-states synchronized by said CPU and MCU thereby producing a series of polarization states mapping out a discrete subset of available polarization states visualized by the Point Care' Sphere for illuminating a target.

3. The apparatus of claim 1 wherein said light source consists of a randomly polarized propagating collimated light beam directed to propagate through a series of independent rotatable optical elements consisting of a rotating linear polarizer and rotating quarter waveplate whereby each rotating element is individually controlled to step through a series of incremental angles over a span ranging from 0° to 180° thereby forming a series of independent polarization states used to illuminate the target.

4. The apparatus of claim 1 wherein said light source consists of a polarized light source filtered by a rotatable variable-phase wave plate, rotating through a series of incremental angles over a span ranging from 0° to 180° thereby forming a series of independent polarization states used to illuminate the target.

5. The apparatus of claim 1 wherein said light source consists of a randomly polarized propagating collimated light beam directed to propagate through a series of independent rotatable optical elements consisting of a rotating waveplate and rotating rotator(for rotating the polarization ellipse) whereby each rotating element is individually controlled to step through a series of incremental angles over a span ranging from 0° to 180° thereby forming a series of independent polarization states used to illuminate the target.

6. The apparatus of claim 1 wherein said multi-state analysis optical circuit consists of a stationary linear polarizer and a rotatable quarter waveplate (QWP) positioned in line with the field of view of the camera to filter the backscattered beam imaged by the camera, the QWP positioned in a rotational bearing positioned by said CPU and MCU to angles ranging incrementally from 0° to 180° by Q integral number of steps, with each step corresponding to one data file producing a total of Q data files making up said data set for calculating the Stokes Parameters on a pixel basis.

7. The apparatus of claim 1 wherein the light source utilized to probe the target consists of wavelengths not in the visible range, including radio frequency bands or terahertz bands for probing metals or plastics or other hard and opaque objects.

8. The apparatus of claim 1 where in the light source utilized to probe targets is in the visible range and consists of a series of color bands for optical sectioning of targets, illumining the target one color or multiple colors at a time to produce unique data set.

9. The apparatus of claim 1 wherein the classified quantitative data produced by analyzing the parametric data set is overlayed onto a pictorial image of the target creating a mask to register pixelated quantitative descriptors with physical locations pertaining to the target

10. The apparatus of claim 1 wherein data analysis is performed by filtering each registered pixelₙₖ n=1 to i, k=1 to j (where i is the number of row pixels and j the column pixels), for each of the N*K Stokes parameters S0ij,S1ij,S2ij,S3ij for maximum values forming a data set Sv(max)=S0ij(max),S1ij(max),S2ij(max),S3ij(max) to calculate the degree of polarization Pij=sqrt((S1ij(max)\hat{ }2)+(S2ij(max)\hat{ }2)+(S3ij(max)\hat{ }2))/+(S0ij(max)) whereby parametric data file Pij is used to classify each pixel or pixel bin based upon degree of polarization.

11. The apparatus of claim 1 wherein data analysis is performed by filtering each registered pixelₙₖ n=1 to i, k=1 to j for each of the N*K data files, for a subset of the four Stokes parameters S0ij,S1ij,S2ij,S3ij using one or more parameters forming numerically derived relations for pixel classification.

12. The apparatus of claim 1 wherein data analysis is performed by forming a subset of the complete parametric data set (Sv for each N*K polarization state) utilizing a subset of Stokes parameters and a subset N*K polarization states, upon which said data analysis is performed.

13. The apparatus of claim 1 wherein data analysis is performed by forming an algorithm utilizing said parametric data consisting of one or more of the four Stokes parameters (S0,S1,S2,S3) or combinations of therein for the series of illumination polarization states, or combination of Stokes parameters, and or data files utilized to calculate the Stokes parameters all utilized to create algorithms based upon photon tissue optical relationships, polarization contrast ratios, degree of polarization, polarization visibility or other numerical relationship for parametric classification of pixels or bin of pixels pertaining registered to physical location of the target.

14. The apparatus of claim 1 wherein data analysis is performed by forming an algorithm acting on the parametric data set, formed by machine learning techniques used to classify pixels or pixel bins registered to physical location of the target.

15. The apparatus of claim 9 wherein said algorithm is used in concert with pictorially derived algorithm acting together on the parametric data set or subset of the parametric set to classify pixels or pixel bins registered to physical location of the target.

16. A method of making an apparatus providing an optical beam of a particular polarization state illuminating a target, providing a means to sequentially step the polarization state to a series of states including linear, circular and elliptical states mapping out a subset of polarization states depicted by the Point Care' Sphere; providing a means for imaging a back scattered optical beam emanating off a target filtered by reconfigurable optical elements producing a series of digital pictures supporting the calculation of the Stokes parameters for each input polarization thereby forming a wavelength spectrum dependent data set, to be analyzed by a trained algorithm or numerically derived analysis for pixel or pixel bin classification on a quantitative basis registered to physical location of the target.
